(19) United States
(12) Patent Application Publication
(10) Pub. No.: US 2020/0268791 A1
(43) Pub. Date: Aug. 27, 2020

(54) A COMPOSITION AND METHOD FOR THE TREATMENT OF ACNE VULGARIS

(71) Applicant: AVI DASCALU, TEL-AVIV (IL)

(72) Inventor: AVI DASCALU, TEL-AVIV (IL)

(21) Appl. No.: 16/464,406

(22) PCT Filed: Apr. 7, 2017
(86) PCT No.: PCT/IB2017/000481

§ 371 Date: May 28, 2019

Related U.S. Application Data
(60) Provisional application No. 62/434,055, filed on Dec. 14, 2016.

(51) Int. Cl. A61K 33/16; A61K 33/04; A61P 17/10

(52) U.S. Cl. CPC A61K 33/16; A61K 9/0014; A61P 17/10; A61K 33/04

(57) ABSTRACT

The present invention is directed to methods and compositions for treatment and reduction of acne vulgaris and related skin disorders, and more specifically, to a composition comprising a combination of aluminum fluoride and sulfur as the sole active agents for the treatment of the acne vulgaris and related skin disorders.

### CROSS-REFERENCE TO RELATED APPLICATION

This application claims priority to U.S. Provisional Patent Application No. 62/434,055 filed on Dec. 14, 2016, the contents of which are incorporated by reference herein for all purposes.

### BACKGROUND OF THE INVENTION

### Technical Field

The present invention provides for compositions and methods of using such compositions for the treatment of acne vulgaris or related skin conditions, and more specifically, to a composition comprising a combination of aluminum fluoride and sulfur as the sole active agents for the treatment of acne vulgaris and related skin disorders.

### Related Art

Acne vulgaris is an inflammatory obstruction of the sebaceous gland caused by an impact of the sebaceous duct, generally due to a genetic predisposition. This genetic switch is activated by hormones, especially after puberty, although acne vulgaris (Latin for ordinary) can appear up to 60 years age old. It appears on sebaceous areas, such as face, chest and upper back.

Plugging of sebaceous gland by hyperproliferation and abnormal differentiation of keratinocytes creates a white or black comedo, depending on its depth (Habif T. Clinical Dermatology, Elsevier, 6th ed., 2015). Together, superficial impacts, named black comedos, and more deeper impacted white comedos are clinically defined as non-inflammatory lesions.

Dermal inflammation is due to sebum entrapped in the lower part of the follicle, which invade the dermis due to follicular cell rupture. Such an intrusion of lipids into the dermal tissue or even deeper causes irritation by lipids, and secondary colonization by microbes such as Propionebacterium acnes (P. acnes) which finally triggers an inflammatory response and cellular infiltrate. These are identified as inflammatory lesions. Clinically, the inflammatory response is perceived as papule, pustule, cyst or nodule (Bolognia Dermatology 3rd ed. Elsevier, 2012.). Acne scoring is defined by the absolute quantity of both inflammatory and non-inflammatory lesions. These types of lesions might induce persistent scars, a predominant reason for treatment of acne. Identical acneiform lesions are caused by chloride, hydrocarbons (Andrews p 90), petrolatum, tar, paraffin, bromide, iodide (Bolognia Dermatology 3rd ed. Elsevier, 2012.), and fluoride (Epstein E., 1976).

Present acne vulgaris pharmaceutical treatments include the use of topicals, such as, salicylic acid, sulfacetamide, clindamycin, erythromycin, retinoids, azelaic acid, benzoylperoxide creams, a combination of aluminum fluoride, sulfur and resorcinol, all of which have been found to be not that effective.

Since the abovementioned treatments did not reach convincing results or instead caused annoying side effects, combinations products were issued, alike clindamycin-benzoyl peroxide, erythromycin-benzoyl peroxide, adapalene-benzoyl peroxide, clindamycin-tretinoin, etc. These combination products did achieve only a slighter efficacy but at the price of higher irritancy rate or other negative side effects.

The relative efficacy of the present known topicals causes non responders of treatment to further drift toward the use oral antibiotics. These oral drugs are in part more efficacious but bear some side effects alike. For example, doxycycline and tetracycline, induce a risk for auto-immune hepatotoxic reaction and lupus like syndrome and might cause a rare but permanent pigmentation. Commonly they cause lower tract infections in female patients. Erythromycin is effective but can cause severe gastric side effects. The use of azithromycin has been reported to cause cardiac side effects.

The use of hormonal combination therapy in women has been tried but not without related cardiovascular complications and lipid disorders. Further, upon ceasing use of such hormones, about a third of the patients exhibit an exacerbation of acne.

The most effective medication is the oral use of isotretinoin, but its use is limited (Rook Textbook of Dermatology, 9th ed.) due to teratogenicity, hepatotoxicity, increase in intracranial pressure, nose bleedings, muscular and joint pains, abnormal liver function, premature epiphyseal closure, decreased bone mineral density and even a relationship to depressive modes.

Thus, in light of the above discussed shortcomings of current treatments it would be advantageous to provide a new combination of drugs that shows effectiveness and safety in the treatment of acne vulgaris.

### SUMMARY OF THE INVENTION

The present invention is directed to methods and compositions for treatment and reduction of acne vulgaris. The present invention is based on the discovery that a composition comprising the combination of aluminum fluoride and sulfur as the sole active agents can be used to treat acne vulgaris. The present invention will be illustrated herein in particular with reference to acne vulgaris but is not restricted thereto.

Accordingly, in one aspect, the present invention provides compositions for treatment of acne, wherein in the composition consists essentially of two active agents, that being aluminum fluoride (AlF3) and sulfur (AlF-S) Additional non-active agent may be included.

In light of the ability of AlF3-Sulfur to treat acne vulgaris, said compositions should be able to be used in treating in addition to acne vulgaris other related skin disorders in mammalian skin and scalp, such as acne rosacea, folliculitis or seborrhea.

In another aspect, the present invention provides for in compositions for the treatment of acne vulgaris, comprising a first active ingredient AlF3 or chemical compounds comprising as an active ingredient aluminum fluoride or combinations of aluminum and fluoride salts which finally release aluminum fluoride in combination with elemental sulfur as a second active agent. The composition according to the present invention comprises AlF3 at a concentration from 0.001-10.0%, preferably between 0.01-5%; and advantageously between 0.2-1.0% by weight. The elemental sulfur is included at concentration from 0.001-30.0%, preferably between 0.1-10%; and advantageously between 0.5-5.0% by weight.

The composition according to the present invention may further comprise additional pharmaceutically and/or cosmetically acceptable compounds.

The active ingredients may be formulated into various pharmaceutically and/or cosmetically compositions, i.e. a solution, a lotion, a tonic, a shampoo, a gel, a mousse, a wax, a stick, a mask, a soap, a moisturizer, a powder, a brilliantine, an aerosol, a pomade, a cream, an ointment, or a paste.

In yet another aspect, the present invention provides for a method for treating or reducing the negative effects of acne vulgaris in a subject, the method comprising:
administering and preferably topically applying to a subject a therapeutic effective amount of a composition consisting essentially of aluminum fluoride and elemental sulfur as the two sole active agent to treat the acne vulgaris, wherein the therapeutic amount is in an amount about 0.2 to 2 grams a day; and
continuing the administration until symptoms of acne are reduced or abated.

In a further aspect of the present invention provides for a method for treatment of acne or related skin condition comprising:
topically administering, to an area of human skin affected by acne or related skin condition a topical dosage form comprising aluminum fluoride and sulfur as sole active agents; and
continuing the administration until symptoms of acne or related skin condition are either relieved or abated.

Yet another aspect of the present invention provides for a topical composition comprising:
aluminum fluoride in a concentration of about 0.01% to about 2% w/w;
sulfur in a concentration of about 0.5% to 5% w/w;
and at least three of the following components selected from the group consisting of:
at least one thickener in a concentration of about 0.5% to about 5% w/w;
at least one preservative in a concentration of about 0.01% to about 2% w/w;
at least one moisturizer in a concentration of about 0.01% to about 5% w/w;
at least one surfactant in a concentration of about 0.1% to about 5% w/w;
and water from about 80% to 97% w/w.

In one aspect, the composition is administered to said subject as related to its clinical response, i.e. preventively from once, twice or thrice weekly to at least once, twice, three times, four times or five times a day. In addition, the composition is administered to said subject over a period of 1 to 12 weeks or longer depending on the subject and severity of acne. Additional preventive treatments may be carried out once-twice weekly as required by the clinical response. The effectiveness of treatment may be monitored, for example, by monitoring the skin of said subject.

In yet another aspect, the present invention provides for a composition that is suitable for topical application. For example, the composition may be in the form of a cream, lotion, paste, gel, liquid or spray. In addition, the composition may be a sustained release composition, for example, for release of the combination of aluminum fluoride and sulfur during 6 hours to 48 hours.

In a still further aspect, the present invention provides for the use of the composition consisting essentially of aluminum fluoride and elemental sulfur in the manufacture of a medicament for the treatment of acne vulgaris.

Other features and advantages of the invention will be apparent from the following detailed description and claims.

### BRIEF DESCRIPTION OF THE FIGURES

FIG. 1 shows Total Lesion Count (TLC) raw data, a change at 3 mo. as compared with start of treatment. TLC results were compared between the Placebo, Triad and AlF-Sulfur groups of patients. The comparison demonstrated a statistically significant difference between groups (p<0.05, Table I, Kruskal-Wallis One Way Analysis of Variance on Ranks).

FIG. 2 shows Investigator Global Scale (IGA), Bar plot success vs. failure for Placebo, Triad and AlF-Sulfur. The raw data for improvement or failure was subjected to a statistical test (Chi square test for trend). Upon performing the test, the differences between groups were statistically significant (Table IV, p<0.05).

### DETAILED DESCRIPTION OF THE INVENTION

The present invention provides a composition to treat acne vulgaris and related skin disorders that is highly successful with low side effects. The present invention provides all these following beneficial effects, including improved clinical acne score for topical use, available to women without teratogenicity; low irritancy and highly tolerable, no phototoxicity and devoid of oral antibiotic or retinoid negative side effects.

As used herein, the term “acne vulgaris” refers generally to the art recognized condition, characterized by blackheads (black spots the size of a pinhead), whiteheads (white spots similar to blackheads), pustules (small pus-filled lesions) and redness and inflammation around eruptions. If acne is severe, cysts (larger, firm swellings in the skin), and abscesses (swollen, inflamed, tender area of infection containing pus) are also visible

As used herein, the term “aluminum fluoride” is intended to refer to the art recognized compound aluminum fluoride, also known as aluminum trifluoride or AlF3 (CAS No 7784-18-1, EINECS No. 232-051-1).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

As used herein, the term “subject” includes warm-blooded animals, preferably mammals, including humans. In a preferred embodiment, the subject is a primate. In an even more preferred embodiment, the primate is a human.

As used herein, the terms “treat,” “treatment” and “treating” include the application or administration of the compositions of the present invention, for example, aluminum fluoride/sulfur compositions, to a subject who is suffering from acne vulgaris with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

As used herein, the term “administering” to a subject includes dispensing, delivering or applying a composition comprising aluminum fluoride and sulfur or consisting of the combination of aluminum fluoride/sulfur as two sole active agents to a subject by any suitable route for delivery of the aluminum fluoride/sulfur combination to the desired location in the subject, including delivery by topical application. Alternatively or in combination, delivery is by intramuscular injection, subcutaneous/intradermal injection, buccal administration, transdermal delivery, topical delivery. Preferably, the compositions of the invention are administered topically, e.g., to the skin of an affected subject.

As used herein, the term “sustained delivery” or “sustained release” is intended to refer to continual delivery of a composition of aluminum fluoride and elemental sulfur in vivo over a period of time following administration, preferably at least several days, a week or several weeks and up to a month or more. In a preferred embodiment, a formulation of the invention achieves sustained delivery for at least about 7, 14, 21 or 28 days, at which point the sustained release formulation can be re-administered to achieve sustained delivery for another 28 day period (which re-administration can be repeated every 7, 14, 21 or 28 days to achieve sustained delivery for several months to years). Sustained delivery of the combination of aluminum fluoride/sulfur can be demonstrated by, for example, the continued therapeutic effect of the aluminum fluoride/sulfur over time.

As used herein, the term “therapeutically effective amount” includes an amount effective, at dosages and for periods of time necessary, to achieve the desired result, e.g., sufficient to treat a subject suffering from acne vulgaris or similar skin conditions. An effective amount of the aluminum fluoride/sulfur combination, as defined herein may vary according to factors such as the state, severity and extent of the condition, weight of the subject, and the ability of the compound to elicit a desired response in the subject. Dosage regimens may be adjusted to provide the optimum therapeutic response. An effective amount is also one in which any toxic or detrimental effects (e.g., side effects) of the aluminum fluoride/sulfur combination are outweighed by the therapeutically beneficial effects.

The aluminum fluoride/sulfur combination is present in the compositions in an amount effective to treat, for example, alleviate, or reduce the effects of acne vulgaris. The aluminum fluoride may be present in the compositions at a concentration of about from 0.001-10.0%, preferably between 0.01-5%; and advantageously between 0.2-1.0% by weight. The elemental sulfur is included at concentration from 0.001-30.0%, preferably between 0.1-10%; and advantageously between 0.5-5.0% by weight.

Alternatively or in combination, the aluminum fluoride may be present as a pharmaceutically acceptable salt. Moreover, alternatively or in combination, the composition may include chemical compounds which react or combine to form and release in vivo aluminum fluoride, for example a combination of aluminum and fluoride salts to achieve the desired therapeutic effect. Various fluoride embodiments, such as sodium monofluorophosphate, sodium fluoride and stannous fluoride are related to the present invention since they may release or combine with aluminum in a mixture.

The compositions of the present invention may further include other agents, for example, inactive carriers. Inactive carriers including, but not limited to, deionized water, cyclomethicone 5, arachidyl alcohol, behenyl alcohol, arachidyl glucoside, montanov 202, cetearyl alcohol, capric caprilic triglyceride, isopropyl palmitate, steareth-2, dimethicon, steareth-20, allantoin, propylene glycol, methylisothiazolinone, Caprylic/Capric acids; medium chain, triglycerides, PEG 400, Pluronic F127, sodium benzoate, and combinations thereof may also be included.

Moreover, the compositions of the invention may further include additional non-active pharmaceutically and/or cosmetically acceptable compounds and/or compositions. Such as:
Surfactants;
Emollients;
Thickening agents;
pH stabilizers;
and
Preservatives.

In a particular embodiment, the aluminum fluoride/sulfur composition is suitable for topical administration and specifically applied to skin affected and showing the effects of acne vulgaris. For example, the composition may be in the form of a gel, liquid or spray to allow, for example, for topical application to the area affected with acne. Alternatively, the composition may be in the form of a solution, lotion, mask, soap, moisturizer, powder, brilliantine, aerosol, pomade, cream, ointment or paste.

The composition can be formulated as a solution, microemulsion, liposome, or other ordered structure suitable for delivery. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prolonged absorption of the compositions can be brought about by including in the composition an agent which delays absorption, for example, monostearate salts and gelatin. Moreover, the compounds of the invention can be administered in a time release formulation, for example in a composition which includes a slow release polymer. The aluminum fluoride/sulfur compositions can be prepared with carriers that will protect the aluminum fluoride against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers. Many methods for the preparation of such formulations are patented or generally known to those skilled in the art.

In one aspect of the invention, the aluminum fluoride/sulfur is applied in admixture with a dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin. While the carrier for dermatological compositions can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration.

Many preparations are known in the art, and include lotions containing oils and/or alcohols and emollients such as hydrocarbon oils and waxes, silicone oils, vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids.

Various types of other ingredients may be present in compositions of the present invention. For example, sunscreens may be included such as those materials commonly employed to block ultraviolet light. Illustrative compounds are the derivatives of PABA, cinnamate and salicylate. The exact amount of sunscreen employed in the compositions can vary depending upon the degree of protection desired from the sun's UV radiation.

Emollients are often incorporated into the therapeutic compositions of the present invention. Levels of such emollients may range from about 0.5% to about 60% w/w, preferably between about 5% and 30% by weight of the total composition. Emollients may be classified under such general chemical categories as esters, fatty acids and alcohols, polyols and hydrocarbons. Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Suitable fatty alcohols and acids include those compounds having from 10 to 20 carbon atoms. Especially preferred are compounds such as cetyl, arachidyl, behenyl, cetearyl, myristyl, palmitic and stearyl alcohols and acids. Among the polyols which may serve as emollients are linear and branched chain alkyl polyhydroxyl compounds. For example, propylene glycol, menthol, bisabolol, sorbitol and glycerin are preferred. Also useful may be polymeric polyols such as polypropylene glycol and polyethylene glycol. Exemplary hydrocarbons which may serve as emollients are those having hydrocarbon chains anywhere from 12 to 30 carbon atoms. Specific examples include mineral oil, lanolin, shea butter, petroleum jelly, paraffin oil, squalene and isoparaffins.

Another category of functional ingredients within the therapeutic compositions of the present invention are thickeners. A thickener will usually be present in amounts anywhere from 0.1% to 20% w/w, preferably from about 0.2% to 10% by weight of the composition. Exemplary thickeners are cross-linked polyacrylate materials. Gums may be employed such as xanthan, carrageenan, gelatin, karaya, pectin and locust beans gum. Under certain circumstances the thickening function may be accomplished by a material also serving as a silicone or emollient. For instance, silicone gums having a viscosity in excess of 10 mPas and esters such as glycerol stearate have dual functionality.

Still further, the therapeutic compositions of the present invention may include preservatives, moisturizers, surfactants, antimicrobials, etc. Preservatives may include tetrasodium ethylene-diamine tetraacetic acid (EDTA), methylparaben, propylparaben, benzophenone-4, methylchloroisothiazolinone, sodium benzoatemethylisothiazolinone, and the like, and mixtures thereof. Preservatives, when used, are typically present in an amount from about 0.01% to 10% weight, preferably about 0.05% to 4% weight, and more preferably, from about 0.1% to 2% weight.

The inclusion of moisturizers may include wheat protein (e.g., laurdimonium hydroxypropyl hydrolyzed wheat protein), hair keratin amino acids, sodium peroxylinecarbolic acid, panthenol, tocopherol (Vitamin E), dimethicone, arachidylglucoside and the like, and mixtures thereof. Moisturizers, when used, are typically present in an amount from about 0.01% to 10% weight, preferably about 0.05% to 1.5% weight, more preferably, from about 0.1% to 1% weight of the composition.

Acceptable surfactants, including both the foaming and non-foaming type, include Lutrol L44, Trolamine®, sodium laureth sulfate, sodium laureth-13 carboxylate, disodium laureth sulfosuccinate, disodium cocoamphodiacetate, glycol stearate, PEG-150 distearate and the like, and mixtures thereof. The surfactant component may be present in an amount from about 0.1% to about 10% weight of the composition.

Any pharmaceutically acceptable antimicrobial agent available to those of ordinary skill in the art may be used in the present compositions including: echinacea, golden seal, benzalkonium chloride, triclosan, benzethonium chloride, iodine, grape seed extract, pomegranate extract, green tea extract or polyphenols, and the like, or combinations thereof. The antimicrobial agent is typically present in an amount from about 0.01% to 2% weight, preferably from about 0.1% to 1.2% weight, and more preferably from about 0.3% to 1% weight of the composition. The antimicrobial agent inhibits the formation, and may further reduce, the presence of microbes that cause redness, inflammation, and irritation of the skin.

The topical skin treatment composition of the invention can be formulated as a lotion having a viscosity of from 4,000 to 10,000 mPas, a fluid cream having a viscosity of from 10,000 to 20,000 mPas or a cream or a gel having a viscosity of from 20,000 to 100,000 mPas or above.

The composition can be packaged in a suitable container to suit its viscosity and intended use by the consumer. For example, a lotion or fluid cream can be packaged in a bottle or a roll-ball applicator, or a capsule, or a propellant-driven aerosol device or a container fitted with a pump suitable for finger operation. When the composition is a cream, it can simply be stored in a non-deformable bottle or squeeze container, such as a tube or a lidded jar.

Generally, in the practice of methods of the invention, the composition is topically applied to the affected skin areas in a predetermined or as-needed regimen either at intervals by application of a lotion or the like, it generally being the case that gradual improvement is noted with each successive application. For example, in use, a small quantity of the composition, for example from 1 to 5 ml, is applied to exposed areas of the skin, from a suitable container or applicator and, if necessary, it is then spread over and/or rubbed into the skin using the hand or fingers or a suitable device.

Because of its ease of administration, a cream, lotion, gel or ointment represents the most advantageous topical dosage unit form, and such forms may be prepared as rinse-off or leave-on products, as well as two stage treatment products for use with other skin cleansing or managing compositions. Each of these forms is well understood by those of ordinary skill in the art, such that dosages may be easily prepared to incorporate the pharmaceutical composition of the invention.

The compositions of the present invention may be administered as necessary to achieve the desired effect and depend on a variety of factors including, but not limited to, the severity of the condition, age and history of the subject and the nature of the composition, for example, concentration of aluminum fluoride/sulfur and/or sustained release capabilities. In various embodiments, the compositions may be administered at least two, three, four, five or six times a day. Additionally, the therapeutic or preventative regimens may cover a period of at least 1 to 24 weeks.

The effectiveness of the treatments described herein can be assessed based on a variety of factors including, for example, the disappearance of small pus-filled lesions, redness and inflammation around eruptions, and cysts or abscesses.
The compositions according to the present invention may further comprise, for example, one or more supplementary pharmaceutically and/or cosmetically active compounds capable of functioning in different ways to enhance the activity of AlF-sulfur and/or to provide other anti-acne advantages, as follows.
Topical antibiotics, alike clindamycin or erythromycin;
Retinoids, alike tretinoin, adapalene, tazarotene and isotretinoin;
Azelaic acid;
Benzoyl peroxide creams;
Dapsone gel;
Astringents, e.g. aluminum chloride, camphor, allantoin, etc;
Antifungals, e.g. azoles, metronidazole, alyllamines, etc;
Nitric oxide precursors such as L-arginine, or compounds that cause the release of nitric oxide, e.g. glyceryl tri-nitrate;
Various deplugging products: alpha-Hydroxy acids, (such as glycolic acid and lactic acid), 5-alpha reductase inhibitors, calamine lotion, salicylic acid, zinc, zinc oxide; and
salicylic acid, sulfacetamide.

The composition according to the present invention may be applied also as part of a physical therapy, e.g. with ultraviolet, blue light spectrum or infrared radiation, of cryotherapy, of ultrasound, etc.

The composition according to the present invention may be prepared, for example, by conventional methods as becomes apparent from the Examples given hereinafter. The present invention will now be illustrated with reference to the following Examples and to Figures and Tables, annexed hereto without being limited by them.

### EXAMPLES

### Example 1

A double blind study was conducted comparing various acne treatments (n=261). Patients were treated up to a 90 days period. Subjects applied twice daily the acne remedies as a gel formulation. A count of Total Lesion Counts (TLC), i.e. inflammatory and non-inflammatory lesions, is presented, comparing between 3 arms: Placebo, Triad (AlF 0.30%, Sulfur 2.5% and Resorcinol at 0.25% U.S. Pat. No. 7,452,556) and AlF-Sulfur (AlF 0.30%, Sulfur 2.5%). Patients did apply one “fingertip unit”, approximately 0.5 grams, evenly over both sides of the face (including the nose), twice a day, no sooner than approximately 8 hours apart, for a total daily dose of 1 g/day. The study medication was required to remain at room temperature.

Following the informed consent process, the patients signed a consent form. Patient's demographics and concomitant medications were recorded, vital signs obtained, and a physical examination was performed. Medical history was recorded to include all ongoing conditions. Patients were instructed to wash their face with non-comedogenic, non-astringent skin cleanser prior to application of the gel to dried skin. Patients did apply the first dose of their assigned treatment under the supervision of the investigative site personnel, and the amount of gel needed to cover the entire face was determined using a provided dosing card. The second application of study gel was no sooner than approximately 8 hours after first dose and no later than 2 hours before the patient goes to bed.

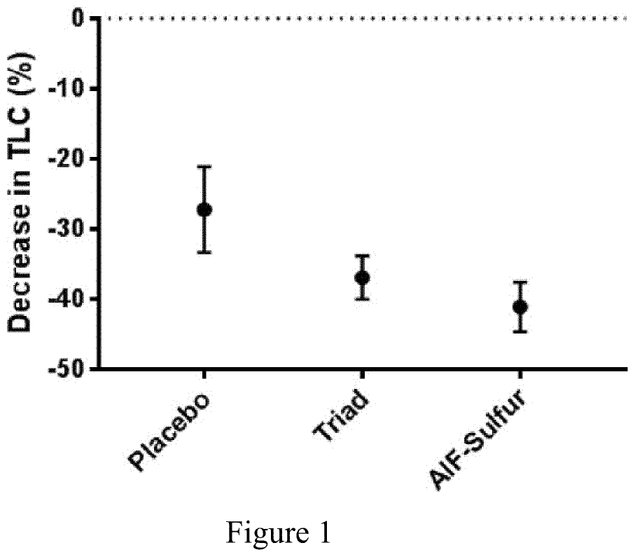

Results are exemplified as a decrease in percentages of TLC between start of treatment and end of treatment for all three groups: Placebo, Triad and AlF-Sulfur, respectively, as Mean+SEM (Figure. 1). A larger decrease in TLC signifies a more efficacious treatment. Table 1 shows the group statistics.

TABLE I
TLC, Group statistics for Placebo, Triad and AlF-Sulfur, respectively.
Test of Kruskal-Wallis One Way Analysis of Variance on Ranks

| | | | |
|---|---|---|---|
| Number of values | 88 | 84 | 89 |
| Minimum | −77 | −97 | −96 |
| Maximum | 452 | 68 | 131 |
| Mean | −27.24 | −36.94 | −41.11 |
| Std. Deviation | 57.28 | 28.42 | 33.24 |
| Std. Error of Mean | 6.106 | 3.101 | 3.523 |
| Lower 95% CI of mean | −39.38 | −43.11 | −48.11 |
| Upper 95% CI of mean | −15.1 | −30.77 | −34.11 |

Unexpectedly, Total Lesion Count (TLC) of a combo formula consisting of only of two of the compounds, i.e. AlF-Sulfur, outperformed the results of the triad of AlF-Sulfur-Resorcinol (Triad) as compared to Placebo (Example 1, FIG. 1, Table I and II).

Ii is highly surprising to find that omitting one anti acne compound, the resorcinol, improves the efficacy of the drug. Resorcinol is classically added to acne remedies and generally recognized as safe and useful, and it is even part of an acne OTC monograph (21CFR333-310 of April 2016). Its omission actually improves the anti-acne drug efficacy, rendering the effect of the AlF-Sulfur as the best outcome.

It is concluded that the superior efficacy of the AlF-Sulfur to the Triad is due to resorcinol deletion. This surprising effect achieved by combining Sulfur and ALF makes it an ideal candidate as an acne drug. Remarkably, an approved and documented anti-microbial compound as resorcinol is hindering efficacy of this particular Triad combo.

Further detailed comparison of active treatments versus placebo did exemplify a significant statistical significance for AlF-Sulfur (p<0.05) as compared to the placebo treated patients. No such significance exists for the Triad treatment (Multiple comparisons by Dunn's Method, SigmaPlot 6.0, Systat Software) as shown below in Table II

### Multiple Comparisons Versus Control Group (Dunn's Method)

TABLE II
TLC, Multiple Comparisons versus
Control Group (Dunn's Method)

| Comparsion | Diff of Ranks | Q | P < 0.05 |
|---|---|---|---|
| ALF-S TLC vs Placebo TLC | 29.675 | 2.615 | Yes |
| Triad TLC vs Placebo TLC | 13.736 | 1.193 | No |

Therefore, it is concluded that as for Total Lesion Counts, the AlF-Sulfur performs superior to the Triad combination of AlF-Sulfur-Resorcinol. Also, it is proved that omitting the use of Resorcinol in the formula improves clinical outcome of Acne Vulgaris.

### Example 2

An Investigator Global Assessment (IGA) score is required as part of measuring improvement criteria (Table III). Investigators did perform a global assessment using the following scale which has been used in studies supporting FDA approval of other treatments for this indication (Thiboutot D M, 2007). Assessments were performed at the start of the study and each month up to 3 months. Data is presented for subjects completing at least 2 mo. of treatment.

TABLE III
IGA scoring by investigator
Table III The score amounts from clear
to severe scores (0 to 4) as follows:

| Grade | Description | |
|---|---|---|
| 0 | Clear | Normal, clear skin with no evidence of acne. |
| 1 | Almost Clear | A few scattered comedones and a few small |
| | | papules (residual hyperpigmentation and |
| | | erythema may be present). |
| 2 | Mild | Easily recognizable; some comedones and some |
| | | papules and pustules (no more than a few |
| | | inflammatory lesions). |
| 3 | Moderate | Many comedones, papules and pustules. One |
| | | nodule may be present. |
| 4 | Severe | Covered with comedones, numerous papules |
| | | and pustules, and few nodules and cysts. |

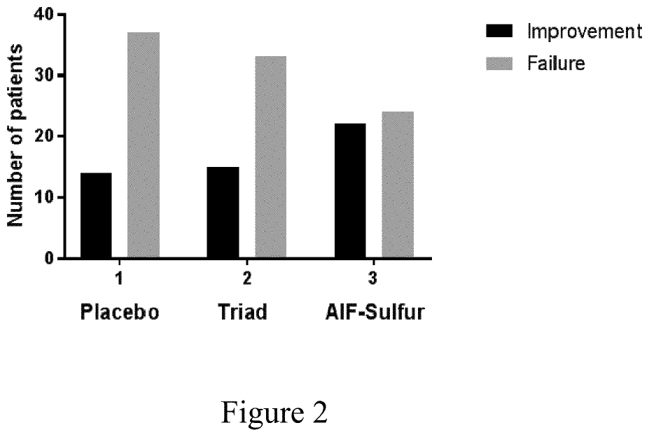

Comparison was used for “success” or “improvement” versus “failure” (Chi square test, GraphPad Prism 7, GraphPad Software). Success is defined as an improvement of at least 2 categories of the IGA before and after treatment, as described in the table. Failure is defined as no improvement of the clinical score. FIG. 2 exhibits the raw data of all three groups, Placebo, Triad and AlF-Sulfur. A gradual disappearance of treatment failures starting with Placebo through triad to AlF-Sulfur is observed, accompanied by a reciprocal gradual increase of the success rate from Placebo to AlF-Sulfur.

Upon comparing IGA improvement of AlF-Sulfur and Triad as compared to Placebo, it was found that AlF-Sulfur outperforms the Triad. Therefore, an independent methodology in a large scale clinical trial did replicate the counting TLC results in a double blinded study (Example 2, FIG. 2, Table IV-VII).

TABLE IV
IGA, statistical data for Placebo, Triad and AlF-S.

| Table Analyzed | Placebo vs. Triad vs. AlF-Sulfur |
|---|---|
| P value and statistical significance | |
| Test | Chi-square test for trend |
| Chi-square, df | 4.313, 1 |
| P value | 0.0378 |
| P value summary | * |
| One- or two-sided | NA |
| Statistically significant (P < 0.05)? | Yes |
| Data analyzed | |
| Number of rows | 3 |
| Number of columns | 2 |

Next, a comparison between AlF-Sulfur and Placebo was performed by a contingency table. Table V, shows the raw data, and Table VI the statistical analysis, thereby exemplify the improvement versus failure for placebo and AlF-Sulfur, at a significance of p<0.05 (Chi square). On the contrary, such a statistical significance was not achieved by a comparison of Triad versus Placebo (P=NS, Chi square, Table VII).

TABLE VI
IGA data, AlF-Sulfur versus Placebo contingency table.

| Table Analyzed | AlF-S vs Placebo |
|---|---|
| P value and statistical significance | |
| Test | Chi-square |
| Chi-square, df | 4.302, 1 |
| z | 2.074 |
| P value | 0.0381 |
| F value summary | * |
| One- or two-sided | Two-sided |
| Statistically significant (P < 0.05)? | Yes |

TABLE VII
IGA data, Triad versus Placebo contingency table.

| Table Analyzed | Triad vs Placebo |
|---|---|
| P value and statistical significance | |
| Test | Chi-square |
| Chi-square, df | 0.1723, 1 |
| z | 0.4151 |
| P value | 0.6781 |
| P value summary | ns |
| One- or two-sided | Two-sided |
| Statistically significant (P < 0 05)? | No |

It is concluded, by these results, that AlF-Sulfur performs superior and is more efficacious as an acne vulgaris treatment than Triad as measured by a double blind methodology.

### Example 3

Formulation of Anti-Acne Gel

| Material and Grade | % |
|---|---|
| Purified Water, USP | 94.150 |
| Aluminum Fluoride Trihydrate | 0.493 |
| Methylparaben, NF | 0.150 |
| Sulfur | 2.500 |
| Propylparaben, NF | 0.050 |
| Carbopol 980 NF Polymer | 1.000 |
| Cyclomethicone 5, NF | 0.500 |
| Lutrol ® L44 (Polaxamer 124, NF) | 0.500 |
| Trolamine, NF (Triethanolamine-99 NF) | 0.657 |
| TOTAL | 100.0000 |

### Example 4

Formulation of Anti-Acne Gel

| Material and Grade | |
|---|---|
| Purified Water, USP | 95.400 |
| Aluminum Fluoride Trihydrate | 0.493 |
| Methylparaben, NF | 0.150 |
| Sulfur | 1.250 |
| Propylparaben, NF | 0.050 |
| Carbopol 980 NF Polymer | 1.000 |
| Cyclomethicone 5, NF | 0.500 |
| Lutrol ® L44 (Polaxamer 124, NF) | 0.500 |
| Trolamine, NF (Triethanolamine-99 NF) | 0.657 |
| TOTAL | 100.0000 |

### Example 5

Formulation of Anti-Acne Cream

| Material and Grade | % |
|---|---|
| Purified Water, USP | 94.696 |
| Aluminum Fluoride Trihydrate | 0.247 |
| Methylparaben, NF | 0.150 |
| Sulfur | 2.500 |
| Propylparaben, NF | 0.050 |
| Carbopol 980 NF Polymer | 1.000 |
| Cyclomethicone 5, NF | 0.500 |
| Lutrol ® L44 (Polaxamer 124, NF) | 0.200 |
| Trolamine, NF (Triethanolamine-99 NF) | 0.657 |
| TOTAL | 100.0000 |

### Example 6

Formulation of Anti-Acne Gel

| Material and Grade | |
|---|---|
| Purified Water, USP | 91.650 |
| Aluminum Fluoride Trihydrate | 0.493 |
| Methylparaben, NF | 0.150 |
| Sulfur | 5.000 |
| Propylparaben, NF | 0.050 |
| Carbopol 980 NF Polymer | 1.000 |
| Cyclomethicone 5, NF | 0.500 |
| Lutrol ® L44 (Polaxamer 124, NF) | 0.500 |
| Trolamine, NF (Triethanolamine-99 NF) | 0.657 |
| TOTAL | 100.0000 |

### REFERENCES

The contents of all references cited herein are hereby incorporated by reference herein for all purposes.
Habif T., Clinical Dermatology, 6th Edition. Elsevier, 6th ed., 2015. Acne, Rosacea and related disorders, Ch. 7. pp 218.
Dascalu Avi. U.S. Pat. No. 7,452,556. Compositions for the treatment of pilosebaceous gland inflammations comprising aluminum fluoride.
Epstein E. Arch. Dermatol Fluoride toothpastes as a cause of acne-like eruptions, 1976 July, 112(7): 1033-4
Bolognia J, Jorizzo J. and Schaffer J. Elsevier, eds. Bolognia Dermatology 3rd edition, 2012. Thiboutot D M, Zaenglein A L. Acne Vulgaris, Ch. 36, pp 545.
Griffiths C et al. eds, Rook's Textbook of Dermatology 9th edition. 2016. Tidman M J and Smith C H. Principles of systemic therapy. Ch. 19., 19.38.
21CFR333-310 of April 2016, FDA, drugs for human use Topical antimicrobial drug products for over-the-counter human use, Topical Acne Products, Acne active ingredients.

1. A composition to treat acne vulgaris in a subject, the composition comprising a combination of aluminum fluoride and sulfur as sole active agents in a therapeutic amount to treat acne vulgaris.

2. A composition for treatment of acne, wherein the composition consists essentially of aluminum fluoride and elemental sulfur as the sole active agents to treat acne.

3. The composition of claim 2, further comprising non-active agents.

4. The composition of claim 2, wherein the aluminum fluoride is in a concentration of about 0.2-1.0% w/w and the elemental sulfur is in a concentration of about 0.5-5.0% w/w.

5. The composition of claim 2, wherein the composition is formulated for topical application.

6. The composition of claim 5, wherein the composition is a lotion, cream, ointment or paste.

7. The composition of claim 3, wherein the non-active agents are selected from the group carriers, surfactants, emollients, thickening agents, pH stabilizers and preservatives.

8. A topical composition comprising:
aluminum fluoride in a concentration of about 0.01% to about 2% w/w;
sulfur in a concentration of about 0.5% to 5% w/w;
and at least three of the following components selected from the group consisting of:
at least one thickener in a concentration of about 0.5% to about 5% w/w;
at least one preservative in a concentration of about 0.01% to about 2% w/w;
at least one moisturizer in a concentration of about 0.01% to about 5% w/w;
at least one surfactant in a concentration of about 0.1% to about 5% w/w;
and water from about 80% to 97% w/w.

9. A method for treating or reducing the negative effects of acne vulgaris or related skin conditions in a subject, the method comprising:
topically applying to a subject a therapeutic effective amount of a composition consisting essentially of aluminum fluoride and elemental sulfur as the only active agents to treat the acne vulgaris; and
continuing topically applying until symptoms of the acne vulgaris are reduced or abated.

10. The method of claim 9, wherein the composition further comprises non-active agents.

11. The method of claim 9, wherein the aluminum fluoride is in a concentration of about 0.2-1.0% w/w and the elemental sulfur is in a concentration of about 0.5-5.0% w/w.

12. The method of claim 9, wherein the composition is formulated as a lotion, cream, ointment or paste.

13. The method of claim 10, wherein the non-active agents are selected from the group carriers, surfactants, emollients, thickening agents, pH stabilizers and preservatives.

14. The method of claim 9, wherein the composition is applied at least once a day for a period of 1 to 12 week.

15. The method of claim 9, wherein the therapeutic effective amount is about 0.2 to 2 grams a day.

16. (canceled)
